(19) United States
(12) Patent Application Publication
(10) Pub. No.: US 2024/0099280 A1
(43) Pub. Date: Mar. 28, 2024

(54) INSTALLATION FOR REARING ARTHROPODS IN MULTI-TIERED MODULES

(71) Applicant: Innovafeed, Nesle (FR)

(72) Inventors: Bastien Oggeri, Paris (FR); Audrey Schuller, Nogent-Sur-Marne (FR); Antonin Tixier, Nanterre (FR)

(21) Appl. No.: 18/263,782

(22) PCT Filed: Feb. 4, 2022
(86) PCT No.: PCT/FR2022/050223

§ 371 Date: Aug. 1, 2023

(30) Foreign Application Priority Data
Feb. 5, 2021 (FR) FR2101144

(51) Int. Cl. A01K 67/033

(52) U.S. Cl. CPC A01K 67/033; A23K 10/20

(57) ABSTRACT

An arthropod rearing installation comprises modules including multi-tiered blocks each having a plurality of transverse trays superimposed and joined by irremovable spacers delimiting at least one lateral slot between each of the transverse trays, and means for moving the modules, the means comprising a support interacting directly with the bottom of the modules without interposition of a pallet. The modules form a multi-tiered assembly without moving components and that cannot be disassembled without being destroyed. The installation further comprises shelving for the storage of arthropod rearing modules and equipment for episodically performing at least one rearing treatment. The installation further comprises a means for emptying the multi-tiered monolithic modules by tilting the assembly thereof, without any separation of the tiers of the modules.

### CROSS REFERENCE TO RELATED APPLICATIONS

This application is a national phase entry under 35 U.S.C. § 371 of International Patent Application PCT/FR2022/050223, filed Feb. 4, 2022, designating the United States of America and published as International Patent Publication WO 2022/167770 A1 on Aug. 11, 2022, which claims the benefit under Article 8 of the Patent Cooperation Treaty to French Patent Application Serial No. FR2101144, filed Feb. 5, 2021.

### TECHNICAL FIELD

The present disclosure relates to the field of the large-scale rearing of arthropods, in particular of insects for the purposes of producing food for animals or humans.

### BACKGROUND

Insects have a certain number of features that make them well suited for use in animal feed. Insects in fact have a high protein content, while being rich in other beneficial nutrients such as fats, minerals and vitamins. The levels of protein concentration in insect meals intended for animal feed vary between 55% and 75%. Insects are characterized by a higher food conversion rate and can therefore become a very valuable feed source for farm animals. Insects are a natural component of the feed of animals such as carnivorous fish (for example, insects can provide up to 70% of the food needs of trout) or poultry. Moreover, these products also have a well-balanced nutritional profile to meet human food needs.

These considerations have led to the development of the automated mass production of food from the rearing of arthropods, and more particularly of insects, in industrial sites organized into complementary spaces specialized in the hatching, rearing, collection of mature animals and their treatment to extract the compounds of interest. These industrial sites must be optimized to offer the best health guarantees, and to ensure suitable climatic conditions to allow optimal and reproducible larvae growth in all seasons.

To install such production sites, US 2020253176 discloses an insect larvae rearing system, the system comprising a container for a feed for insect larvae, rearing modules configured to handle a plurality of trays of larvae and to supply the feed to the trays. The feed is supplied from a container to each of the rearing modules. The container comprises a tray handling system arranged to receive trays or a stack of trays at a loading/unloading point and to move the trays or the stack of trays, and a feed delivery system configured to deliver feed received from outside the container to each tray repeatedly. The stack may comprise wheels or casters on the bottom of the trays, the bottom-most tray of a stack of interlocked trays or racks. The tray handling system may comprise ball transfer units and/or rollers. The tray handling system may comprise guide rails. The rearing module may be arranged such that the trays or the stacks of trays are insertable into and removable from the tray handling system using a forklift truck, on rollers or casters or another form of transport system.

This solution is designed for the production of larvae intended to supply an adjacent avian farm. The production is carried out in containers, sheet metal buildings wherein stacks of trays are enclosed that may be extracted from this building-container by a forklift truck. When the stack of trays is in a container, it is not possible to perform any treatment. It is necessary to open the container, to extract the stack by moving the other stacks, which prevent access thereto, to extract the stack with a conveyor to remove it from the container, and to move it into another building by passing through an exterior space. This solution is not suitable for larva rearing requiring several operations within the same building, without passing through the exterior space.

Patent KR200491810 describes an insect rearing box having a quadrangular enclosure shape wherein the upper part is open. The insect rearing box has a central inner lower surface inclined downward. The boxes are stacked. A groove penetrating the inner lower surface and the outer lower surface is provided at the center of the lower surface of the insect rearing box.

This concave shape is not appropriate because it leads to a concentration of the larvae and nutrients in the central zone, and does not allow homogenization of the rearing conditions over the entire available surface.

Patent CN110178797 describes a device for cultivating insects with several pallets, comprising at least two rearing boxes and a metal frame. A movable flap is provided with a hinge allowing this flap to be opened.

This device consists of an assembly of parts made of different materials, with articulated parts that are poorly suited to intensive use. Indeed, this system seems not to be very robust with respect to repeated handling, and there is a risk of locking of the moving parts that can lead to poor emptying of these rearing boxes.

Patent application WO2014171684 relates to a multi-layered box assembly for growth and storage, the purpose of which is to provide a multi-layered box assembly for growth and storage to enable the maximum utilization of a restricted space wherein various types of living things grow.

British patent GB1109015 describes a single-piece container formed from foamed plastic and comprising a base portion having walls that stand up thereon, the base portion having a convex upper surface and integral reinforcing ribs formed on its lower surface. The container may have the shape of an open box provided with means for facilitating stacking, adequate strength being ensured by providing thickened corner posts, which can support similar containers when stacked above as well as constituting spaced feet to be used when the container is standing on a planar surface.

This solution is not satisfactory because the handling of individual trays is tedious and slow, implying a very high number of handling operations compared to the number of handling operations necessary to manage multi-tiered modules.

In general, the solutions of the prior art are not totally adapted to continuous usage, for automated industrial production leading to a very large number of handling operations and displacements from one device to another, and for being left at rest during rearing phases without unnecessarily cluttering the air conditioned space wherein the various equipment items are arranged.

### BRIEF SUMMARY

In order to remedy the problems of the prior art, the present disclosure relates, according to its most general accepted form, to an arthropod rearing installation including multi-tiered rearing modules each having a plurality of transverse trays superimposed and joined by spacers delimiting at least one lateral slot between each of the transverse trays, and means for moving the rearing modules comprising a support interacting directly with the bottom of the modules, without interposition of a pallet. Each of the rearing modules forms a monolithic multi-tiered assembly without moving components. The installation further comprises shelving for the storage of the arthropod rearing modules and equipment for episodically performing at least one rearing treatment. The shelving has parallel supports whose spacing is greater than ⅓ of the width of the lower transverse tray and less than the width of the lower transverse tray.

In additional embodiments, the present disclosure related to an arthropod rearing module intended for such an installation. The arthropod rearing module comprises a monolithic multi-tiered block without moving parts having a plurality of transverse trays that are superimposed and joined by spacers delimiting at least one lateral slot between each of said transverse trays.

In yet further embodiments, the present disclosure relates to a method for rearing arthropods, in which a rearing medium is deposited on trays of multi-tiered monolithic rearing modules each having a plurality of transverse trays that are superimposed and joined together by spacers delimiting at least one lateral slot between each of the transverse trays. The rearing medium is inoculated by pouring arthropods in an early growth stage. Episodic displacements of the rearing modules are performed, without disassembly, and in a closed loop, between shelving able to store the arthropod rearing modules, and equipment items to perform at least one rearing treatment by displacement means comprising a support directly interacting with the bottom of the modules, without interposition of a pallet. The method further comprises, before each new treatment, checking the quality of a content of the rearing modules extracted from racks. The rearing modules whose content is not compliant during the quality control step are directed toward an evacuation zone comprising a tipper dedicated to a step of emptying and transferring to waste.

### BRIEF DESCRIPTION OF THE DRAWINGS

The present disclosure will be better understood on reading the following description, with reference to the appended drawings showing non-limiting embodiments, in which:

FIG. 1 shows a perspective view of a rearing module;

FIG. 2 shows a median cross-sectional view of the rearing module;

FIG. 3 shows a view of the tray before assembly of the rearing module;

FIG. 4 shows a side view of a rearing module;

FIG. 5 shows a median cross-sectional view of the rearing module arranged on a rack;

FIG. 6 shows a median transverse cross sectional view of the rearing module on the support of a shuttle;

FIG. 7 shows a median cross-sectional view of the rearing module arranged on a conveyor belt;

FIG. 8 shows a schematic view of a rearing module at the beginning of emptying;

FIG. 9 shows a schematic view of the rearing module in an intermediate emptying situation;

FIG. 10 shows a schematic view of the rearing module in an emptying situation;

FIG. 11 shows a schematic view of the rearing module in a filling situation; and

FIG. 12 shows a schematic view of the rearing cycle.

### DETAILED DESCRIPTION

The industrial production of feed originating from the rearing of arthropods and—in the example described in a non-limiting manner—of insects is done in largely automated installations intended to create optimal conditions for massively allowing the passage from the evolutionary stage of the egg, the egg capsule or the neonatal larva, to the larva (or maggot or apteran), to nymph or pupa, to the adult insect.

Typically, these installations are organized in buildings each comprising shelving for storing the rearing modules loaded with an inoculated rearing medium, in a rearing-favorable climatic atmosphere, as well as handling equipment for episodic treatments. The objective is to avoid transfers outside the building as much as possible by performing all the treatments required for rearing within the same building, with an optimization of the space leading to providing a storage zone and a treatment zone, as well as transfer means, remaining in the same building, between the storage zone and the treatment zone. To this end, the present disclosure proposes a multi-tiered module designed for optimized cultivation of the larvae and for the occasional transfer to treatment equipment inside the same building.

The present disclosure aims to optimize large-scale production by simplifying handling both to save time and to increase production, to allow for the use of reliable and economical equipment, as well as to guarantee sanitation conditions that reduce the risks of infection or of pathogen development.

Production Cycle

Unlike most of the industrial production solutions of the prior art, which operate linearly, the installation according to the present disclosure operates in a closed cycle, in particular as it relates to the circulation of the rearing modules. All of the treatments from filling of a rearing module until the mature insects are recovered and the rearing module is reinjected into the rearing circuit, take place in a closed loop, according to a cycle of a few days (typically 6 to 20 days), without requiring transfer to or from the outside of the building.

This cycle, executed without transfer outside the treatment site, comprises a succession of steps:
storing the rearing modules filled with the rearing medium inoculated with larvae in shelving of a rearing site in a controlled atmosphere (temperature, hygrometry, controlled light, controlled atmosphere as regards the chemical and/or biological composition);
transferring to treatment equipment;
emptying the rearing module for recovery of the rearing medium and insects by tilting it;
washing and rinsing the emptied rearing module;
injecting the rearing medium into the rearing module;
inoculating larvae from neonates or young larvae in the pre-growth phase;
resuming the aforementioned storage step, the rearing modules being permanently recycled (except for occasional breakage).

The efficiency of the installation is therefore particularly conditioned by:
the configuration of the rearing modules;
the operating mode of some of the handling and transportation equipment.

In the building, contrary to the teaching of document US2020253176, the multi-tiered modules are stored on storage shelving, from which they are removed by an internal transfer system between the storage shelving and one of the treatment equipment items.

Growth Steps

Optionally, the cycle is broken down into a pre-growth step followed by an incubation step.

During the pre-growth step, the rearing module (100) is loaded with a first rearing medium suitable for young larvae, and is inoculated with larvae in the neonate (newborn) stage. When these young larvae have reached a predetermined reference size, the rearing module is emptied and the young larvae are recovered to inoculate a new rearing module loaded beforehand with a new rearing medium suitable for this second stage of maturity.

Rearing Modules

In order to optimize both the storage space and the handling equipment, the rearing modules according to the present disclosure have a multi-tiered configuration that allows storage on racks and handling in a single rigid, monolithic block with no moving parts, not requiring any pallet or additional support to be moved.

As used herein, the term “monolithic” means a rigid assembly with non-separable parts, except by deliberate disassembly, forming a single block that can be handled in its entirety without the possibility of handling some of its parts separately (except in the event of disassembly or destruction) and therefore without moving parts. The use of moving parts may indeed pose problems of robustness for the system and may lead to risks of locking.

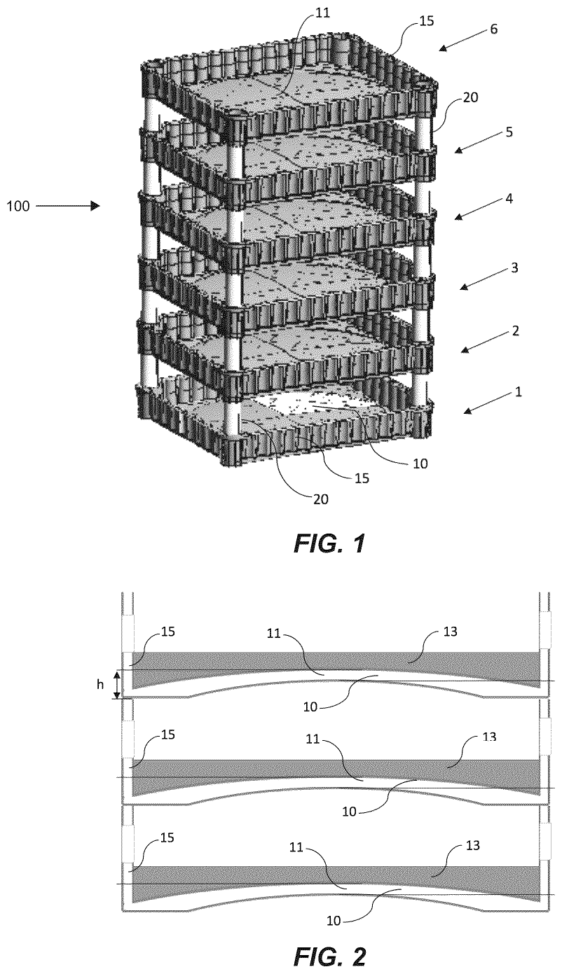

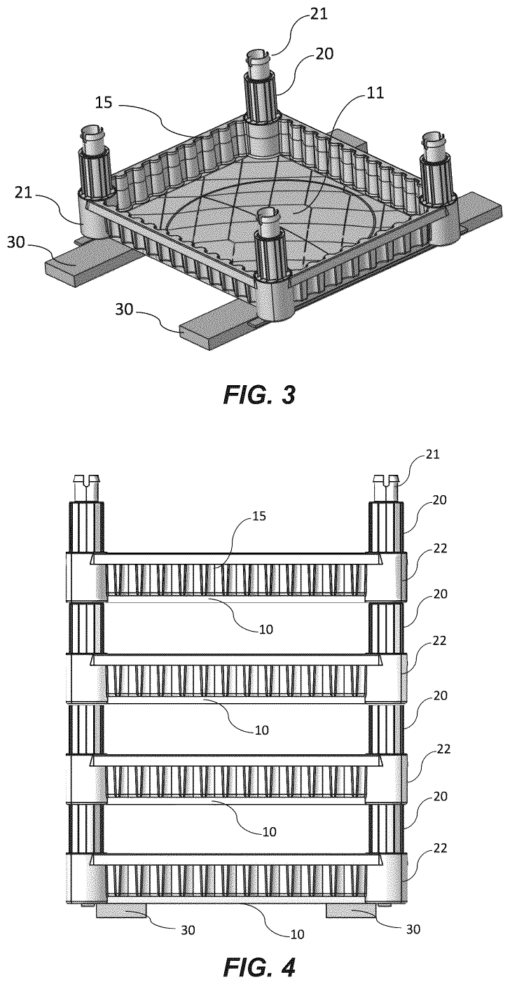

In the non-limiting example described with reference to FIGS. 1 to 4, the module has six tiers (1 to 6). Each of the tiers has a curved transverse bottom (10) surrounded by a border comprising flutes (15). The consecutive tiers are separated by spacers (20). The cross section of the module is typically 1200×1000 mm, and the height of the borders (15) is 160 mm. The elements are made by injection of plastic.

The modules are thus made up of alternating multi-tiered monolithic blocks of trays whereof the vertical edges have a determined height, for example, 160 mm, and voids separating the transverse plane defined by the rims of these vertical edges and the bottom of the next tray, the interval being, for example, 172 mm. This configuration allows minimization of these heights without compromising:
for the trays:
   the containable substrate volume;
   the absence of overflow during displacements;
   absence of leaked larvae;
for the voids between trays:
   the circulation of air;
   the introduction the substrate injection pipes;
   the introduction of the larvae inoculation chutes.

The modules are formed by an assembly of parts that are then definitively linked, without moving parts, in order to have great robustness compatible with repeated handling operations with automated equipment.

The borders (15) have flutes to reinforce rigidity and strength when the module is loaded, while reducing the weight of the module. The bottom (10) of the trays also has reinforcing ribs for stiffening the rearing module.

The bottoms (10) have a curved shape with a central zone (11) of hemispherical shape with a circular cross section having a diameter of 600 mm, and an elevation at the center of 50 mm. The rearing medium (13) is deposited on the bottom (10). The convex shape of this central zone (11) allows limiting of the deflection, better distribution of the material deposited on the tray (10) and increased deflection tolerance (in order to allow the satellites (30) to pass under the bottom tray of the stack at the time of storage/retrieval).

The rearing trays are assembled to form monolithic modules of six trays by force-fitting the spacers (20) located in the corners (22) with a space of 172 mm between two consecutive trays (10). The elements are connected only by the four spacers and do not rest on one another on whole edges.

The modules cannot be disassembled without deliberate action. In the non-limiting example described, the spacers are forcibly clipped between the trays in a non-reversible manner by deformable clip-fastening members (21).

The spacers (20) are configured to ensure, between two consecutive trays, a lateral slot of a height of between 0.5 and 2 times the height of the edges (15), for example, a lateral slot of 172 mm between two consecutive trays (10) of a module, in order to ensure good circulation of air, and to allow the pouring of the content for emptying the module, as well as the filling as will be explained below.

Handling of the Modules

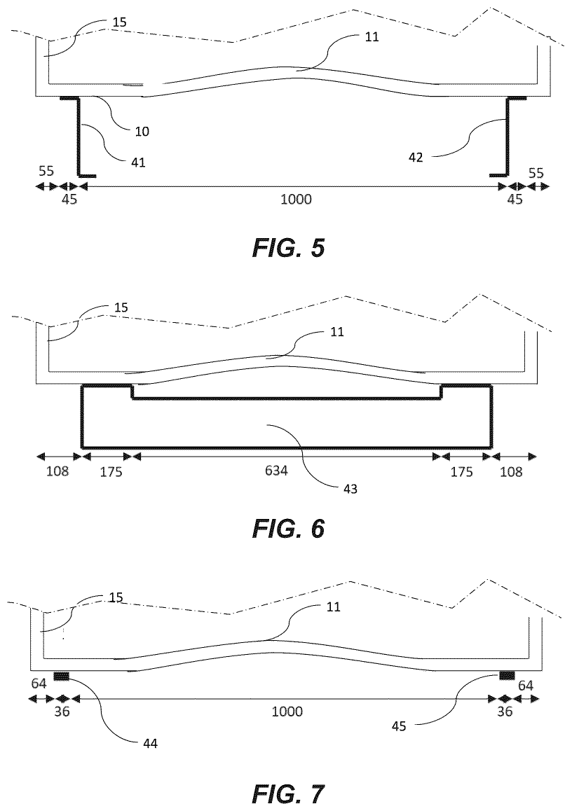

FIGS. 5 to 7 show the handling modes of the modules. The bottoms of the modules rest on racks in a climate-controlled space having supports (41, 42), for example, rails, whereupon it bears on either side of the convex zone (11). These rails (41, 42) are spaced apart to allow the passage of the support (43) positioned under the bottom of the rearing module, without any intermediate pallet, ensuring the removal of the modules or their insertion into a rack. The spacing of these rails is also defined to ensure good stability for the rearing modules (100) during their storage in the climate-controlled space. This spacing must be greater than ⅓ of the width of the lower transverse tray (10) and less than the width of the lower transverse tray (10). The support (43) extends a mobile satellite moving in a perpendicular direction of a guided shuttle to ensure the displacement of the modules to a conveyor and to place them thereon. A plurality of shuttles and satellites allows all the modules to be moved to conveyors. The conveyors then move the modules between the various handling and rearing stations. The width of the conveyors corresponds to the width of the rails (41, 42) of the racks.

Emptying of the Rearing Modules

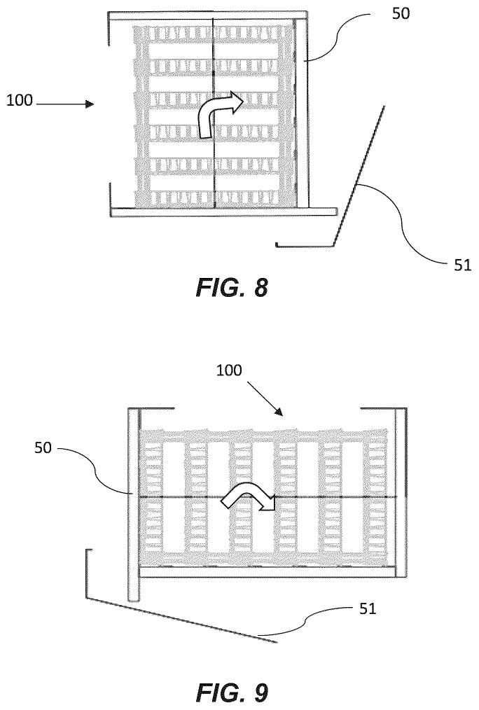

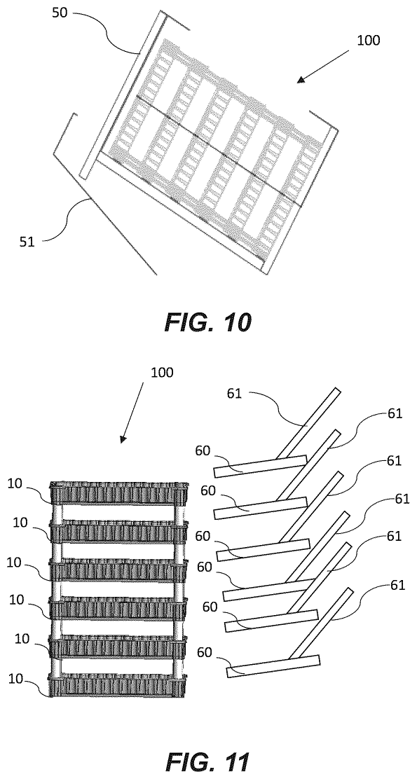

FIGS. 8 to 10 show the process of emptying the modules after maturation of the insects. The module (100) is deposited in an emptying equipment item comprising a frame (50) and a tray (51) that are secured and actuated by pivoting about a transverse axis. During storage in the shelving and during the displacement, the modules (100) are placed vertically, the trays being horizontal. In order to unload the rearing medium and the larvae, the module is tilted by a rotation angle of 120° to 150°, and preferably from 130° to 140°, to allow the contents of the trays to be poured by gravity onto a ramp (51), passing through the slots formed between the edges (15) and the adjacent tray owing to the spacers (20). Optionally, several modules can be emptied simultaneously.

Filling the Rearing Modules

FIG. 11 shows the process of filling the modules (100). It is carried out by a system of ramps (61) pouring the rearing medium onto trays (60) and/or a system of similar ramps pouring the young larvae onto trays (60). This system is positioned between a waiting position where the ramps are separated from the module (100), and a filling position where the ramps (60) are introduced into the module (100), between the trays (10) and passing through the slots freed by the spacers between the consecutive trays. In the example described, the system of ramps (61) is stationary, and the rearing modules (100) are moved using a shifter to allow the filling. Optionally, several filling modules (100) can be filled simultaneously by a system of multiple ramps (61), for example, 3 three series of 6 ramps (61).

The injection involves filling the empty modules (100) with rearing medium from the preparation zone of the rearing medium. The injection line simultaneously fills three juxtaposed modules (100) with a controlled amount and/or composition of rearing medium. The injection line comprises 18 metering pumps pouring their contents into ramps (60).

The inoculation involves depositing insect doses in the trays (10) of the multi-tiered modules (100). These doses are partially conveyed by gravity to the trays (10) and a blowing of compressed air allows the doses to be expelled into the rearing modules and makes it possible to avoid the accumulation of material in the lower part of the inoculation chutes.

The rearing modules are translated on the side via a shifter (conveyor on a rail), which transports the three modules (100) constituting the batch. This shifter moves on an axis perpendicular to the axis of the handling circuit and allows each tray of the rearing modules (100) to be brought under its rearing medium injection nozzle (60), then its larvae inoculating nozzle. These nozzles are located above each tray (10) of the modules (100), they therefore enter the modules (100).

The shifter is shifted to the start of injection position. Position sensors send the information as soon as the rearing modules are sufficiently shifted (that is as soon as the trays (10) of the modules (100) begin to be under the injection nozzles. The module (100) is moved during the injection sequence in order to distribute the rearing medium in the rearing modules (from the start of injection position to the end of injection position. The inoculation is carried out at the end of injection, then the module (100) is replaced in the non-shifted position.

This method allows proper distribution of the rearing medium over all of the trays (10) of the modules (100).

Nature of the Treatments Performed by the Equipment

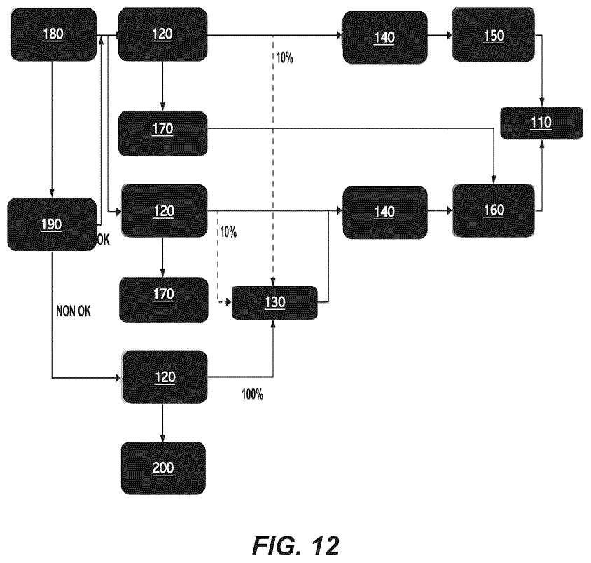

The installation according to the present disclosure allows performance of a treatment cycle applied to each module or group of modules by episodic outputs according to a predetermined sequence of the storage and rearing racks, according to a sequence shown in FIG. 12.

The main treatments are described below. The essence of the cycle involves preserving the rearing modules in the racks provided in a climate-controlled space (step 110). As previously explained, upstream, the matured rearing modules (100) are emptied by the tipper (step 120) and, if need be, are washed (step 130). The empty rearing modules (100) thus prepared are subject to a step of injecting rearing medium (140) followed by a step of inoculation (150) with young neonate larvae, or with larvae having reached a reference size (160). These young larvae injected in step (160) either come from the hatching phase or from a sieving step (170). The sieving is itself fed by a hopper receiving the contents poured into the tipper during the step of emptying (120) the rearing modules extracted from the racks.

Furthermore, before each new treatment, the rearing modules extracted from the racks by batches of three rearing modules are subject to quality control (180) by weighing, measuring the temperature or other rearing parameters, visual inspection (190) and/or optical control by a camera and an automatic image interpretation system.

The rearing modules (100) whose content is not compliant is directed toward a non-compliant discharge zone comprising a tipper dedicated to a step of emptying (120) and transferring to waste (200).

Weighing on the shuttle and optionally measuring the temperature (step 180): at each batch outlet, the module is weighed on the shuttle and the temperature under each tray of each of the rearing modules is measured. The mass of the batch is compared to a threshold mass. If the mass of the batch is less than the threshold value, the batch is considered to be compliant and is sent as intended onto the handling circuit. If it is greater than the threshold value, the batch is considered to be non-compliant and will be sent to the inspection zone. The temperature may constitute an indicator complementary to the mass in order to verify the compliance.

Weighing by rearing module at the handling circuit inlet: The batches arriving on the handling circuit are weighed module (100) by module (100) on a weighing table. This weighing of the rearing module (100) allows a more accurate compliance verification than weighing on the shuttle. For each module (100), the measured mass is compared to a threshold mass. If the mass is less than the threshold value, the module is considered to be compliant. Otherwise, the module is considered to be non-compliant and is sent to the non-compliant discharge zone so that its content may be destroyed.

Non-compliant discharge zone (step 200): The non-compliant branch is used to destroy the content of the rearing modules that is declared non-compliant. These rearing modules are emptied by tilting by a tipper, the operation of which is described above. Their content is emptied, evacuated and ground.

Triple tipper (step 120): when a batch arrives in front of the compliant tipper, the compliant tipper returns the entire batch in order to collect its content by performing several tipping operations, for example, three consecutive tipping operations, to ensure complete emptying, then rests. The module (100) is then sent to the injection and inoculation station.

Weighing at the handling circuit outlet: at the handling circuit outlet, the modules (100) are again weighed one by one.

Handling circuit outlet: the module (100) is sent to storage.

Operator compliance checks: an operator checks the batches that are detected as non-compliant by the shuttle following a measured mass greater than a threshold mass in order to decide on their subsequent treatment. This difference may actually be due to a growth abnormality. The operator performs a visual and tactile check to verify the growth state of each tray of the rearing modules (100).

1. An arthropod rearing installation, comprising:
multi-tiered rearing modules each having a plurality of transverse trays superimposed and joined by spacers delimiting at least one lateral slot between each of the transverse trays; and
displacement means for moving the rearing modules comprising a support interacting directly with the bottom of the modules without interposition of a pallet;
wherein,
each of the rearing modules forms a monolithic multi-tiered assembly without moving components;
the installation further comprises shelving for the storage of the arthropod rearing modules and equipment for episodically performing at least one rearing treatment; and
the shelving has parallel supports whose spacing is greater than ⅓ of the width of the lower transverse tray and less than the width of the lower transverse tray.

2. The arthropod rearing installation of claim 1, wherein the trays have a curved central zone and the shelves have parallel supports whose spacing is between the diameter of the central zone and the width of the lower transverse tray.

3. The arthropod rearing installation of claim 2, wherein the parallel supports are rails spaced apart to allow passage of a support positioned under the bottom of the rearing module, without any intermediate pallet, so as to enable the removal of the modules or their insertion into a rack.

4. The arthropod rearing installation of claim 3, wherein the support extends a mobile satellite moving in a perpendicular direction of a guided shuttle to enable the displacement of the rearing modules to a conveyor and to place them thereupon.

5. The arthropod rearing installation of claim 1, wherein the displacement means are configured to handle the rearing modules as is in a closed loop, between the shelving and the equipment for episodically performing at least one rearing treatment without exiting the installation.

6. The arthropod rearing installation of claim 1, further comprising a means for filling the rearing modules by a supply via a set of ducts pouring a flow of rearing medium and/or larvae onto each of the trays of the multi-tiered rearing modules.

7. The arthropod rearing installation of claim 1, further comprising a means for emptying the multi-tiered monolithic rearing modules by tilting the assembly thereof, not requiring any moving component or any separation of the tiers of the modules.

8. An arthropod rearing module configured for an installation according to claim 1, of the module comprising a monolithic multi-tiered block without moving parts having a plurality of transverse trays that are superimposed and joined by spacers delimiting at least one lateral slot between each of the transverse trays.

9. The arthropod rearing module of claim 8, wherein at least one tray of the plurality of transverse trays has a curved central zone with a diameter smaller than the spacing of the parallel rails of the installation, the width of the at least one tray being greater than or equal to the spacing of the parallel rails.

10. The arthropod rearing module of claim 8, wherein the module comprises an assembly that is not reversible without destroying the plurality of transverse trays, and wherein at least one of the ends of the spacers has a deformable clip-fastening member.

11. The arthropod rearing module of claim 10, wherein each of the trays of the plurality of transverse trays has a curved transverse bottom surrounded by a border comprising flutes.

12. The arthropod rearing module of claim 11, wherein the bottom of each tray of the plurality of transverse trays has reinforcing ribs for stiffening the rearing module.

13. A method for rearing arthropods, comprising:
depositing a rearing medium on trays of multi-tiered monolithic rearing modules each having a plurality of transverse trays that are superimposed and joined together by spacers delimiting at least one lateral slot between each of the transverse trays;
inoculating the rearing medium by pouring arthropods in an early growth stage; and
episodically displacing the rearing modules, without disassembly, and in a closed loop, between shelving able to store the arthropod rearing modules, and equipment items to perform the at least one rearing treatment by displacement means comprising a support directly interacting with the bottoms of the modules, without interposition of a pallet;
wherein the method further comprises, before the at least one rearing treatment, checking the quality of the content of the rearing modules extracted from the shelving, the rearing modules whose content is not compliant during the checking of the quality being directed toward an evacuation zone comprising a tipper configured to empty the content of the respective rearing module and transfer the emptied content to waste.

14. The method of claim 13, further comprising filling said the rearing modules by inserting, into the slots of the rearing modules, ramps fed by metering systems to deposit the rearing medium and/or arthropods at an early growth stage onto the trays.

15. The method of claim 13, further comprising emptying the rearing modules by tilting the rearing modules to pour the contents of the trays onto a collection means.

16. The arthropod rearing installation of claim 1, wherein the parallel supports are rails spaced apart to allow passage of a support positioned under the bottom of the rearing module, without any intermediate pallet, so as to enable the removal of the modules or their insertion into a rack.

17. The arthropod rearing installation of claim 16, wherein the support extends a mobile satellite moving in a perpendicular direction of a guided shuttle to enable the displacement of the rearing modules to a conveyor and to place them thereupon.
